SEQUENCE LISTING (8 sequences)

SEQ ID NO: 1 - DNA, 68 bp, Artificial Sequence
  synthetic nucleotide primer
gcaaaagtggtcccagaaaccttgagatagaattccagtggtctaacaagggaggtggaggtggagct

SEQ ID NO: 2 - DNA, 74 bp, Artificial Sequence
  synthetic nucleotide primer
tcgatttaagaaacctctcaaacaaaaacaaaaaatgaagtggtggtttaggccccagcggccgcagcagcacc

SEQ ID NO: 3 - DNA, 21 bp, Artificial Sequence
  synthetic nucleotide primer
tacagtttgaaaatttgccgg

SEQ ID NO: 4 - DNA, 25 bp, Artificial Sequence
  synthetic nucleotide primer
tgaggacaacaaaatgttacaaatg

SEQ ID NO: 5 - DNA, 70 bp, Artificial Sequence
  synthetic nucleotide primer
tcagctgctggacatgctcagttggtaaagccgggggagaccattctgacttaccaatgcttaatcagtg

SEQ ID NO: 6 - DNA, 72 bp, Artificial Sequence
  synthetic nucleotide primer
tatattgctctaataaatttttggcgcgccggccaattaggcccgggcggttcaaatatgtatccgctcatg

SEQ ID NO: 7 - DNA, 71 bp, Artificial Sequence
  synthetic nucleotide primer
tctgattaaatttaattaatattatatttttagaatcttctgaatatttaccctcttgggttatcaagagg

SEQ ID NO: 8 - DNA, 73 bp, Artificial Sequence
  synthetic nucleotide primer
ttagttgactgtcagctgtccttgctccaggatgctgtttttgacaacggtaattcctaatttttgttgacac

(19) United States
(12) Patent Application Publication
(10) Pub. No.: US 2017/0280718 A1
(43) Pub. Date: Oct. 5, 2017

(54) HERBICIDAL AND FUNGICIDAL COMPOSITIONS AND THEIR USES

(71) Applicant: The Regents of the University of California, Oakland, CA (US)

(72) Inventors: Georgia Drakakaki, Davis, CA (US); Natasha Worden, Davis, CA (US)

(21) Appl. No.: 15/509,687

(22) PCT Filed: Sep. 9, 2015
(86) PCT No.: PCT/US2015/049245

§ 371 Date: Mar. 8, 2017

Related U.S. Application Data
(60) Provisional application No. 62/048,583, filed on Sep. 10, 2014.

(51) Int. Cl. A01N 43/54; C07C 211/52; C07D 239/42; C07C 211/56; A01N 33/26; C07C 243/22; A01N 43/40; A01N 33/06; A01N 31/16; C07D 213/77; C07C 323/20

(52) U.S. Cl. CPC A01N 43/54; C07D 213/77; C07C 211/52; C07D 239/42; C07C 211/56; C07C 323/20; C07C 243/22; A01N 43/40; A01N 33/06; A01N 31/16; A01N 33/26

(57) ABSTRACT

Described herein are compounds of Formulas I-XIII and agrochemically acceptable salts thereof having herbicidal and fungicidal activity. Also disclosed herein are herbicidal and fungicidal compositions, including compounds of Formula I-XIII or agrochemically acceptable salts thereof, and methods of controlling unwanted vegetation or fungus using compositions including the compound of Formula I-XIII or an agrochemically acceptable salt thereof.

### CROSS-REFERENCE TO RELATED APPLICATIONS

This application claims priority benefit of U.S. Provisional Application No. 62/048,583, filed Sep. 10, 2014, which is herein incorporated by reference.

### STATEMENT AS TO RIGHTS TO INVENTIONS MADE UNDER FEDERALLY SPONSORED RESEARCH AND DEVELOPMENT

This invention was made with government support under grant number 1258135, awarded by the National Science Foundation. The government has certain rights in the invention.

### BACKGROUND OF THE INVENTION

Herbicides and fungicides are needed to control unwanted growth of vegetation and fungi. As plants and fungicides develop resistance to currently used herbicides and fungicides, development of new compounds becomes necessary. The present invention addresses these and other needs.

Plant cell expansion and anisotropic cell growth is driven by vacuolar turgor pressure and by cell wall extensibility, which in a dynamic and restrictive manner directs cell morphogenesis. Cellulose is the major load-bearing component of the cell wall and is thus a major determinant for anisotropic growth. Cellulose is made up of beta-1,4-linked glucan chains that may aggregate to form microfibrils holding 18-36 chains. In contrast to cell wall structural polysaccharides, including pectin and hemicellulose, which are synthesized by Golgi localized enzymes, cellulose is synthesized at the plasma membrane (PM) by cellulose synthase (CESA) complexes (CSCs). The CESAs are the principle catalytic units of cellulose biosynthesis and are in higher plants organized into globular rosettes. For its biosynthetic function, each CSC requires a minimum of three catalytic CESA proteins. Cellulose synthase complexes (CSCs) at the plasma membrane (PM) are aligned with microtubules and direct the biosynthesis of cellulose. The mechanism of the interaction between CSCs and microtubules (MTs), and the cellular determinants that control the delivery of CSCs at the PM, are not yet well understood.

### BRIEF SUMMARY OF THE INVENTION

On the basis of observations that cellulose microfibrils align with cortical microtubules (MTs), and that MT disruption leads to a loss of cell expansion, it was hypothesized that cortical MTs guide the deposition and therefore the orientation of cellulose (Green, 1962; Ledbetter and Porter, 1963; Baskin, 2001; Bichet et al., 2001; Sugimoto et al., 2003; Baskin et al., 2004; Wasteneys and Fujita, 2006). Confocal microscopy of CESA fluorescent fusions has advanced our understanding of CESA trafficking and dynamics. CSCs are visualized as small particles moving within the plane of the PM with an average velocity of ˜200-400 nm min⁻¹. Their movement in linear tracks along cortical MTs (Paredez et al., 2006) supports the MT-cellulose alignment hypothesis.

Disclosed herein are compounds having herbicidal and fungicidal activity and having general Structure I:

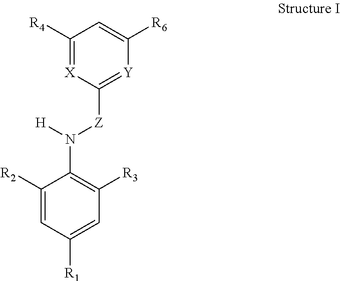

In Structure I, X and Y independently are CH or N; Z is NH or CH₂, R₁ is H or CF₃; R₂ and R₃ independently are Hi or NO₂; R₄ and R₅ independently are H, CH₃ or CF₃.

Compounds having herbicidal and fungicidal activity and having general Structure I include, but are not limited to, compounds of Formulas I-V:

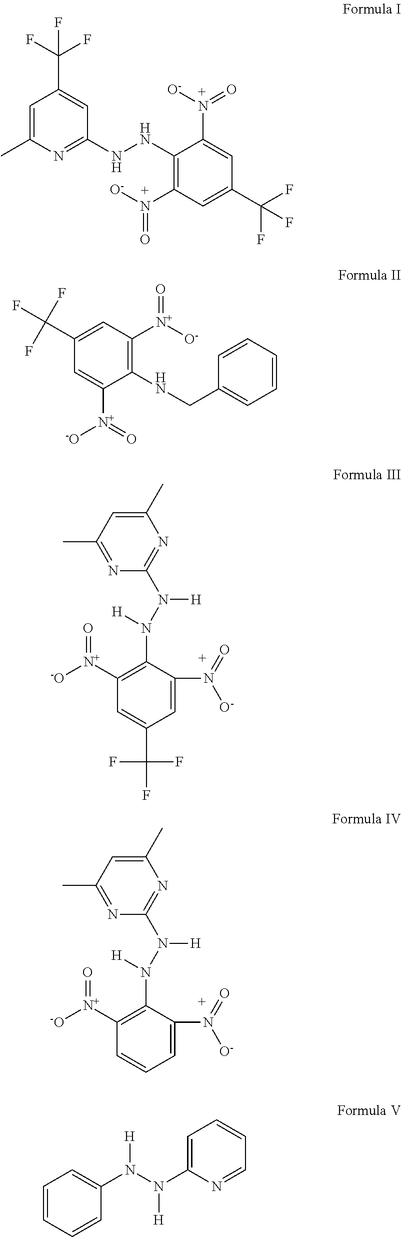

Also disclosed herein are compounds having herbicidal and fungicidal activity and having general Structure II:

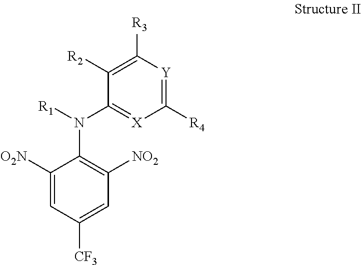

In Structure II, X is CR_{X} or N; Y is CR_{Y} or N; R₁ is H or NH₂; and R_{X}, R_{Y}, R₂, R₃, and R₄ independently are H, CH₃, Cl, or CF₃.

Compounds having herbicidal and fungicidal activity and having general Structure II include, but are not limited to, compounds having Formulas VI-VIII:

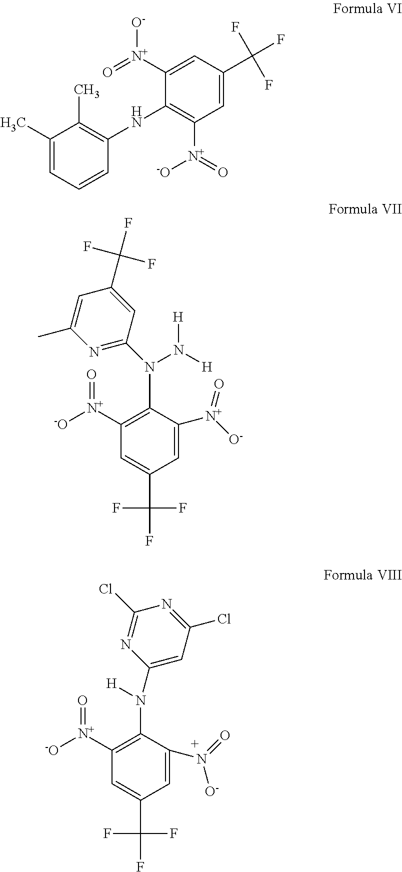

Other compounds have been discovered that also have herbicidal and fungicidal activity. Those compounds include the compounds of Formulas IX-XIII:

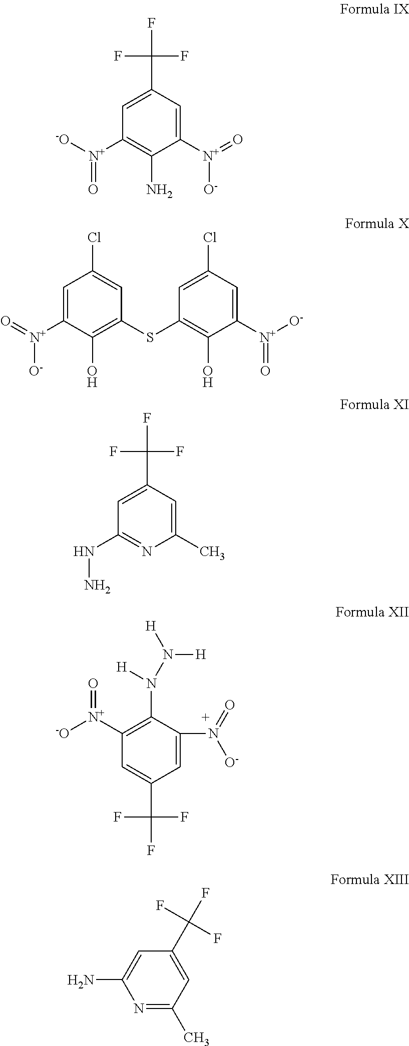

The molecule of Formula I, known as 1-[2,6-dinitro-4-(trifluoromethyl)phenyl]-2-[6-methyl-4-trifluoromethyl)pyridin-2-yl]hydrazine, or alternatively as ACIMVAXZ, herein also is referred to by the name CESTRIN, specifically inhibits cellulose deposition, alters anisotropic growth of Arabidopsis hypocotyls, and induces radial swelling. Distribution and mobility of fluorescently labeled cellulose synthases (CESAs) were monitored in living cells of Arabidopsis under chemical exposure to characterize its subcellular effects. CESTRIN reduces the velocity of PM CSCs and causes their accumulation in the cell cortex. CSC associated proteins, KORRIGAN1 (KOR1) and POM2/CELLULOSE SYNTHASE INTERACTING1 (CSI1), were differentially affected by CESTRIN treatment, indicating different forms of association to the PM CSCs. KOR1 accumulated in bodies similar to CESA, however POM2/CSI1 dissociated into the cytoplasm. In addition, microtubule stability was altered without direct inhibition of microtubule polymerization, suggesting a feedback mechanism caused by cellulose interference. The specificity of CESTRIN was assessed using a variety of subcellular markers for which no morphological effect was observed. The association of CESAs with vesicles decorated by the trans-Golgi network localized SNARE protein, SYP61. was increased under CESTRIN treatment, implicating SYP61 compartments in CESA trafficking. In addition to the above action, CESTRIN inhibits Phytophora capsisi growth. The unique properties of CESTRIN, compared to known cellulose synthase inhibitors, afford novel avenues to study and understand the mechanism under which PM associated CSCs are maintained, interact with microtubules and dissect their trafficking routes, and study cellulose deposition both in plant and oomyces.

The current understanding of cellulose synthesis suggests that CESAs are assembled into CSCs in either the endoplasmic reticulum (ER) or the Golgi apparatus, and trafficked by vesicles to the PM. The presence of CESAs in isolated Golgi and vesicles from the trans-Golgi network (TGN) has been established by proteomic studies. Their localization at the TGN has been corroborated by electron microscopy and colocalization with TGN markers, such as VHA-a1, SYP41, SYP42, and SYP61. A population of post Golgi compartments carrying CSCs, referred to as MASCs or SmaCCs, may be associated with MTs or actin filaments and are thought to be directly involved in either CSC delivery to, or internalization from, the PM.

In addition to the CESAs, auxiliary proteins have been identified that play a vital role in the cellulose synthesizing machinery. These include COBRA, the endoglucanase KORRIGAN1 (KOR1), and the recently identified CELLULOSE SYNTHASE INTERACTING PROTEIN-1/POM2 (CSI1/POM2). The latter protein functions as a linker between the cortical microtubules and CSCs as genetic lesions in CSI1 result in lower incidents of co-alignment between CSCs and cortical MTs. Given the highly regulated process of cellulose biosynthesis and deposition, it can be expected that many more accessory proteins are participating in the delivery of CSCs and their interaction with MTs. Identification of these novel CSC-associated proteins can ultimately provide clues for mechanisms behind cell growth and cell shape formation.

Arabidopsis mutants with defects in the cellulose biosynthetic machinery exhibit loss of anisotropic growth, which results in organ swelling. This phenotype may be used as a diagnostic tool in genetic screens to identify cellulose biosynthetic and CSC auxiliary proteins. Chemical inhibitors complement genetic lesions to perturb, study, and control the cellular and physiological function of proteins. A plethora of bioactive small molecules have been identified and their analytical use contributes to our understanding of cellulose biosynthesis and CESA subcellular behavior, excellently reviewed. Small molecule treatment can induce distinct characteristic subcellular CESA patterns that can be broadly grouped into three categories. The first is characterized by the depletion of CESAs from the PM and their accumulation in cytosolic compartments, as observed for the herbicide isoxaben (N-[3-(1-Ethyl-1-methylpropyl)-5-isoxazolyl]-2,6-dimethyoxybenzamide), CGA 325′615 (1-cyclohexyl-5-(2,3,4,5,6-pentafluorophe-noxyl)-1λ4,2,4,6-thiatriazin-3-amine), thaxtomin A (4-nitroindol-3-yl containing 2,5-dioxopiperazine), and AEF150944 (N2-(1-ethyl-3-phenylpropyl)-6-(1-fluoro-1-methylethyl)-1,3,5-triazine-2,4-di-amine). The second displays hyper accumulation of CESAs at the PM, as seen for the herbicides dichlobenil (DCB) (2,6-dichlorobenzonitrile) and indaziflam (N-((1R,2S)-2,3-dihydro-2,6-dimethyl-1H-inden-1-yl)-6-(1-fluoroethyl)-1,3,5-triazine-2,4-diamine). The third exhibits disturbance of both CESAs and MTs, and alters CESA trajectories at the PM, as exemplified by morlin (7-ethoxy-4-methylchromen-2-one). Novel compounds inducing a phenotype combining CESA accumulation in intermediate compartments and disruption of CSC MT interactions can contribute to both the identification of the accessory proteins linking CSCs with MTs and the vesicular delivery mechanisms of CESAs.

A novel cellulose deposition inhibitor, the small molecule CESTRIN, has been identified and characterized. The molecule affects the localization pattern of CSCs and their interacting proteins in a unique way. The induction of cytoplasmic CESTRIN bodies might provide further clues for trafficking routes that carry CESAs to the PM.

Thus, the novel pharmacologically active compound, CESTRIN, acting as a cellulose deposition and trafficking inhibitor, was identified and its unique mode of action was investigated in detail. CESTRIN specifically alters the trafficking of CSCs and their interacting proteins, enriching the CSC population in SYP61 compartments, and affords novel avenues to study and understand the mechanism under which PM associated CSCs are maintained, interact with MTs, and identify exocytic routes that deliver them to the PM.

Accordingly, compounds described herein include the compounds of Formulas I, II, III, IV, V, VI, VII, VIII, IX, X, XI, XII, XIII and agrochemically acceptable salts thereof. These compounds have herbicidal and fungicidal activity and are useful alone or in combination with other herbicidal and/or fungicidal compounds in compositions as described herein.

Also described herein are herbicidal and fungicidal compositions including one or more of the compounds of Formulas I, II, III, IV, V, VI, VII, VIII, IX, X, XI, XII, XIII or an agrochemically acceptable salt thereof.

Also described herein are methods of controlling unwanted vegetation or fungus using compositions including one or more of the compounds of Formulas I, II, III, IV, and V. VI, VII, VIII, VIIII, X, XI, XII, XIII or an agrochemically acceptable salt thereof.

### BRIEF DESCRIPTION OF THE DRAWINGS

FIGS. 1A-B are micrographs showing localization patterns of CSCs in DMSO and CESTRIN treated plants.

FIGS. 1C-D are time-projected images of translocation tracks of CDCs in DMSO and CESTRIN treated plants.

FIG. 1E is a chart showing average velocity of particles in a treated sample as compared to a control sample.

FIG. 2 are micrographs of different subcellular markers treated with CESTRIN.

FIG. 3A-C are chart and images showing bacterial and yeast growth in media containing CESTRIN or DMSO.

FIG. 4A-B illustrate inhibition of cell elongation and growth in CESTRIN treated plants as compared to DMSO treated plants. It demonstrates a concentration depended inhibition of plant growth by CESTRIN treatment.

FIG. 4C-D demonstrates shape morpohological changes in CESTRIN treated plants as compared to DMSO treated plants.

FIGS. 5A-B and 5D-E are micrographs showing localization patterns of KOR1 or POM2/CSI1 in DMSO and CESTRIN treated plants.

FIGS. 5C and 5F are graphs of velocities of KOR1 and POM2/CSI1 labeled particles of DMSO and CESTRIN treated plants.

FIG. 6A-B are micrographs showing localization patterns of CESA and microtubules in DMSO and CESTRIN treated plants.

FIGS. 7A-C are micrographs and growth curves showing the difference of CESTRIN treatment on cytoskeleton compared to the microtubule depolymerization drug Oryzalin.

FIG. 7D shows in-vitro polymerization of tubulin under 15 μM CESTRIN compared to DMSO and Taxol.

FIGS. 8A-B are micrographs showing an increase in colocalization between CSCs and SYP61 in Arabidopsis.

FIGS. 9A-B are charts comparing hypocotyl growth of ixr1-1, and prc1-1, in CESTRIN compared to DMSO and on media containing 4 nM isoxaben or DMSO.

FIG. 10 provides charts showing the result of root growth analysis of plants treated with various compounds described herein.

FIG. 11 shows inhibition of phytophtora capsici growth in media supplemented with CESTRIN.

### DETAILED DESCRIPTION OF THE INVENTION

Novel cellulose deposition and trafficking inhibitor, CESTRIN, which specifically alters the trafficking of CSCs and their interacting proteins, enriching the CSC population in SYP61 compartments, has been identified. CESTRIN also inhibits Phytophtora capsici growth. CESTRIN affords novel avenues to study and understand the mechanism under which PM associated CSCs are maintained, interact with MTs, and identify exocytic routes that deliver them.

Although recent studies have identified a molecular component that mediates the interaction between CESAs and MTs, many unknown players potentially await discovery. In addition, the cellular determinants that control CSC delivery to, and internalization from, the PM remain ill understood. Such novel components could be critical for regulating the stability and activity of CSCs and may reveal new aspects of anisotropic cell growth. A compound, CESTRIN, which alters the trafficking of CSCs and their associated proteins CSI1/POM2 and KOR1, leading to MT instability and a reduction in cellulose content, has been identified. CESTRIN did not affect the localization patterns of a variety of endomembrane compartments, including ER, Golgi, TGN, and vacuole, demonstrating that the subcellular phenotype is not the result of broad cell toxicity. Strikingly, neither general secretion nor cytosolic clathrin compartments were affected, indicating that CESTRIN's mode of action does not affect indiscriminately endocytic or secretion pathways but rather specialized pathways involved in CESA delivery.

Exposure to CESTRIN increased the colocalization of SYP61 vesicles with CESA, suggesting that CESTRIN bodies are enriched in the syntaxin. Mannitol treatment is thought to tether MT to SmaCCs. Corroborating our findings, mannitol induced SmaCCs only colocalized partially with SYP61 among different TGN markers tested. Moreover, proteomic analysis of SYP61 vesicles has established that they contain CESAs involved in primary cell wall biosynthesis and KOR1, suggesting a role of SYP61 in trafficking of CSCs. A recent TGN proteomic analysis identified CESA1 in VHA-a1 fractions, however neither CESA3/6 nor KOR1 could be detected. A plausible explanation might be that SYP61 defines a population that is distinct from VHA-a1 vesicles involved in the delivery of CSCs.

Cellulose synthase inhibitors (CBIs) influence the trafficking and dynamics of CESAs or MT. Small molecules such as isoxaben, CGA, or mannitol deplete CSCs from the plasma membrane, leading to their subsequent accumulation in SmaCCS/MASCS. CESTRIN is unique in causing CSC accumulation in CESTRIN bodies while concurrently affecting MT organization. The way CESTRIN influences the stability of MTs is markedly different from that observed for CBIs and oryzalin. Application of oryzalin at a concentration depolymerizing MTs, has no significant effect on the velocity and localization of CSCs; however, extended oryzalin treatment results in a complete removal of microtubules and a uniform distribution of CSCs at the PM. Further, under MT depolymerization conditions no dissociation of POM2 with CSCs is observed, despite their localization in less defined trajectories. The only previously described effect of oryzalin in CSC trafficking is in combination with other CBIs. In contrast to oryzalin, which inhibits tubulin polymerization in vitro at concentrations of 5 μM, CESTRIN application at a concentration inducing CESTRIN bodies (15 μM), does not inhibit in vitro tubulin polymerization. This suggests that MT instability or depolymerization are not the primary effects of the small molecule, but rather a feedback mechanism, through an intermediate component associating with both cellulose synthases and MT. The CBIs morlin and cobtorin (4-[(2-chlorophenyl)-methoxy]-1-nitrobenzene) affect cytoskeleton organization but do not cause CSCs accumulation in intermediate bodies. The distinct subcellular phenotypes caused by CESTRIN compared to oryzalin, morlin, and cobtorin illustrate that CESTRIN has a markedly different mode of action, featuring an altered trafficking of extended CSCs and causing their accumulation in intermediate “CESTRIN induced bodies,” while affecting MT-CSC interaction.

Further corroborating that CESTRIN affects cellulose synthesis is the fact that seedlings grown on the compound display a ˜30% reduction in cellulose. In addition, in vitro data show that CESTRIN does not act on microtubule polymerization, which supports a role for CESTRIN in cellulose synthesis rather than microtubule formation. The notion of feedback disruption from cell wall to cytoskeleton organization is supported by genetic studies in which it was shown that mutations in the genes encoding the glucanase KOR1 and CESA6 (kor1-3, prc1-20 and jiaoyao1) resulted in changes of MT organization. It is plausible that CESTRIN's inhibition of the CSCs provides feedback to the microtubules which in turn leads to a dis-organized or collapsing MT array.

CESTRIN may target a link between CSCs and MTs. Studies show that the two CESA interacting proteins POM2/CSI1 and KOR1 are affected by CESTRIN; however, subtly different behaviors were observed for the two. The nature of these proteins might give clues about their subcellular behavior after CESTRIN treatment. CSI1/POM2 is currently the most well characterized protein that serves as a linker between MT and CSCs. In vitro assays demonstrated that it interacts both with MT and CESAs involved in primary cell wall synthesis, while in planta studies have shown that it colocalizes with CESAs while traveling along trajectories aligned with MT CSCs. In addition, genetic lesions in csi1 null mutants exhibit both CSCs and cortical microtubule defects.

The glucanase KOR1 interacts with CESAs involved in the primary cell wall formation and colocalizes with CESAs at the PM following linear trajectories. Moreover, KOR1-GFP is present in SmaCCs/MASCS, at the Golgi, TGN, and late endosome compartments. Under CESTRIN treatment, KOR1-GFP displays subcellular patterns similar to that of GFP-CESA3; both localize in bright fluorescence punctae. This observation suggests that the membrane association of CESAs and KOR1 is maintained upon chemical treatment, leading to their enhanced localization in CESTRIN bodies. This is contrasted by partial cytoplasmic localization of CSI1/POM2 upon chemical treatment, which suggests dissociation from CSCs. The fact that CESTRIN targets both proteins associated with CSCs underscores the specificity of CESTRIN towards a pathway controlling the interaction between the two.

Changes in cellulose content suggest that CESA might be the direct target of the small molecule. It is reasoned, however, that though CESA3 is the target for isoxaben, a different mode of action for affecting CESAs is observed for CESTRIN. The sensitivity of the ixr1-1 mutant to CESTRIN, contrasting that of isoxaben resistance, implies that even if a CESA subunit is targeted by CESTRIN, it does not correspond to the ixr1-1 locus on CESA3. A number of mechanisms can account for the cumulative observed behavior of CESTRIN, though it is challenging to ascribe with certainty likelihoods to these. Plausible hypotheses are presented here.

It is possible that the small molecule acts on a linker protein between CSCs and MTs such as the CSI1/POM2 or other not-yet identified proteins involved in this interaction. Alternatively, CESTRIN might affect a signaling mechanism regulating the activity of CSCs, potentially mediated by phosphorylation. It is known that changes in CESA phosphorylation alter their motility and reduce anisotropic growth. Further interactions of MT associated proteins (MAP65s) and MTs can be modulated via phosphorylation by altering protein surface charge. It is hence tempting to suggest that CESTRIN targets phosphorylation; however, on the basis of the observed reduction in cellulose content and the formation of CESTRIN bodies, without affecting MT polymerization in vitro, this seems unlikely. Another possible mechanism is that signaling events may take place that cause accumulation of CSCs into CESTRIN bodies and feedback altering MT stability. Only future studies that identify the target of CESTRIN can conclusively determine which hypotheses are correct, and these efforts are currently under way.

Accordingly, compounds described herein include CESTRIN and agrochemically acceptable salts thereof. These compounds have herbicidal and fungicidal activity and are useful alone or in combination with other herbicidal and/or fungicidal compounds in compositions as described herein.

Also described herein are herbicidal compositions including the compound of Formula I, or an agrochemically acceptable salt thereof. Also described herein are herbicidal compositions including one or more of the compounds of Formulas II-XIII, or agrochemically acceptable salts thereof.

Also described herein are fungicidal compositions including the compound of Formula I, or an agrochemically acceptable salt thereof. Also described herein are fungicidal compositions including one or more of the compounds of Formulas II-XIII, or agrochemically acceptable salts thereof.

Also described herein are methods of controlling unwanted vegetation or fungus using herbicidal compositions including one or more of the compounds of Formulas I-XIII, or agrochemically acceptable salts thereof.

Herbicidal and fungicidal compositions described herein include CESTRIN, the compound of Formula I, one or more of the compounds of Formulas II-XIII, or agrochemically acceptable salts thereof. The compositions may be formulated as solids, including but not limited to, dusts, granulates, coated granules, impregnated granules, and homogeneous granules; as liquids, including but not limited to, solutions, dispersions, emulsions, and aerosols; and/or as concentrates, including but not limited to the listed solids and liquids in a concentration suitable for dilution prior to use as well as wettable powders and pastes. Thus, the composition may be suitable for direct application or may be prepared in concentrated form suitable for dilution prior to use.

Compositions formulated as liquids or liquid concentrates include one or more of the compounds of Formulas I-XIII or agrochemically acceptable salts thereof, and may further include one or more of a solvent, a liquid dispersing media, surfactant, and emulsifier.

When the compositions described herein are used in the form of solutions, the compound of Formula I, II, III, IV, V, VI, VII, VIII, IX, X, XI, XII or XIII, or the agrochemically acceptable salt thereof, is dissolved in suitable organic solvents, mixtures of solvents, or water using methods well known to those skilled in the art. Suitable organic solvents include, but are not limited to, aromatic hydrocarbons, aliphatic hydrocarbons, cycloaliphatic hydrocarbons and mixtures thereof, such as petroleum distillates. In use, the solvents preferably include the active substances in a concentration range of 1 to 20% based on the total weight of the resulting solution, but may include more or less of the active substance as can be determined by one of skill in the art based on intended use.

Emulsifiable liquid concentrates can be prepared by incorporating one or more of the compounds of Formula I-XIII, or agrochemically acceptable salts thereof, and an emulsifying agent in a suitable water-immiscible organic liquid. Such concentrates may be further diluted with water to form spray mixtures in the form of oil-in-water emulsions. In use, the compound of Formula I, II, III, IV, V, VI, VII, VIII, IX, X, XI, XII or XIII, or the agrochemically acceptable salt thereof, preferably is present in amounts ranging from about 1 to about 30 percent by weight of the total composition, but may be present in greater or lesser amounts as can be determined by one of skill in the art based on intended use. Suitable emulsifying agents can be non-ionic, ionic, or blends thereof. Suitable water immiscible organic liquids include aromatic hydrocarbons, aliphatic hydrocarbons, cycloaliphatic hydrocarbons and mixtures thereof, such as petroleum distillates.

Compositions formulated as liquids or liquid concentrates include one or more of the compounds of Formula I-XIII, or agrochemically acceptable salts thereof, and may further include one or more of a solid carrier, dispersing agent, and/or a solvent. Production of the solid herbicidal and fungicidal compositions described herein is carried out in a manner well-known to those skilled in the art by the intimate mixing and grinding of the active substance, with suitable carriers and optionally dispersing agents, and/or solvents that preferably are inert to the active substances.

Suitable carriers include, but are not limited to, bentonite, kaolin, Fuller's earth, silica, talc, chalk, limestone, ground limestone, dolomite, diatomaceous earth, precipitated silicic acid, alkaline earth silicates, sodium and potassium aluminum silicates (feldspar and mica), calcium and magnesium sulfates, magnesium oxide, ground synthetic plastics, fertilizers such as ammonium sulfate, ammonium phosphate, ammonium nitrate, ureas, ground vegetable products such as grain flour, bark flour, sawdust, ground nut shells, cellulose powder, residues of plant extractions, activated charcoal, etc. These carriers can be used separately or in combination. The grain size of the carriers is from about 0.075 mm to 0.2 mm, and may be larger. For dusts the grain size preferably is less than or equal to 0.1 mm. For sprinkling agents the grain size preferably is about 0.075 mm to about 0.2 mm.

In other embodiments, compositions can be formulated as spreadable granules. The spreadable granules can be prepared using any solid diluent known in the art, but preferably are prepared using calcined attapulgite clay as the solid diluent. For granulates the grain size preferably is equal to or greater than 0.2 mm. Dry dispersions can be prepared on herbicidally and/or fungicidally inert carriers, such as vermiculite, peat moss, and the like.

The concentrations of active substances in the solid preparations preferably are about 0.5 wt % to about 90 wt % of the total composition, and more preferably are about 0.5 wt % to about 80 wt %.

Wettable powders and pastes are examples of concentrates of active substances which can be diluted with water to give any desired concentration. They include one or more of the compounds of Formula I-XIII, or an agrochemically acceptable salt thereof, and one or more carriers, and may optionally include solvents, additives that stabilize the active substance, surfactants, dispersing agents, wetting agents, and/or antifoaming agents. The wettable powder concentrates preferably have concentrations in the range of from about 1 to about 75 percent by weight of the compound of Formula I-XIII, or agrochemically acceptable salt thereof. The wettable powder can be dispersed in water or other hydroxylated carrier to form spray compositions.

Suitable carriers for wettable powders and pastes are, for example, those previously mentioned for solid preparations. Suitable solvents include, but are not limited to, alcohols, benzene, xylenes, toluene, dimethyl sulfoxide and mineral oil fractions boiling between 120° and 350° C. The solvents preferably are practically without smell, not phytotoxic, inert to the active substances, and not easily flammable.

The wettable powders and pastes are obtained by mixing and grinding the active substances with carriers in suitable devices until homogeneity is attained, using methods well known to those skilled in the art. The solid particle size in wettable powders preferably is less than or equal to 0.04 mm and, more preferably, is less than or equal to 0.02 mm. The solid particle size in pastes preferably is less than or equal to 0.003 mm.

Other biocidal active substances or agents can be mixed with the described compositions. Thus, in addition to the stated compounds of Formula I-XIII, or agrochemically acceptable salts thereof, the compositions can also contain, for example, insecticides, fungicides, bactericides, fungistatics, bacteriostatics, or nematocides in order to widen the range of action. The compositions described herein can also contain fertilizers, micronutrients, etc.

The novel compounds described herein can be used for treating a desired area with a dust, granular formulation, or spray containing one or more of the compounds of Formula I-XIII, or an agrochemically acceptable salt thereof, as the herbicidally and/or fungicidally active ingredient. Typical of areas which can be treated are crop growing areas in which tolerant crops are being grown and other areas where control of vegetation is desired, such as gravel driveways, clay tennis courts, walks, road shoulders, and the like.

As is well understood in the art, application rates required when the compounds are to be used in the field are greater than those required in the greenhouse. Compositions containing one or more of the compounds of Formula I-XIII, or an agrochemically acceptable salt thereof, can be sprayed, dusted, or spread by methods well known to the art onto the desired area at the rate of around 1.12 to 56 kilogram of active ingredient per hectare, or preferably 1.12 to 36 kilogram of active ingredient per hectare.

The concentration of the compound in these compositions may vary depending on whether the composition is intended for direct application or is a concentrate designed to be subsequently diluted with additional inert carrier, such as water, to produce the ultimate treating composition.

### Examples

### Material and Methods

Plant Materials and Growth:

Arabidopsis seeds were sterilized using 30% (v/v) sodium chlorate in ethanol (absolute) with 30 μL Triton X-100 (Sigma) per 50 mL of solution. Seeds were plated on 0.5× Arabidopsis growth medium (AGM) with phytagar (2.3 g/L Murashige and Skoog minimal organics medium, 10 g/L sucrose and 8 g/L phytagar), and cold vernalized for 48 hours at 4° C. in the dark. Plates were transferred to a 24° C. growth chamber, exposed upright to light for 3 hours and etiolated in the dark for 3 days prior to chemical treatment and further examination.

Transgenic lines expressing MAN-YFP (Nebenfuhr et al., 1999), HDEL-GFP (Nelson et al., 2007), VAMP711-YFP (Geldner et al., 2009), NTPP-RFP (Rosado et al.), THE1-GFP (Hematy et al., 2007), CLC-GFP (Konopka et al., 2008), VHA-RFP (Dettmer et al., 2006), VTI12-YFP (Geldner et al., 2009), CFP-SYP61 (Robert et al., 2008), sec-GFP (Goh et al., 2007), CESA6-YFP (Paredez et al., 2006), TUB-GFP (Nakamura et al., 2004), EB1-GFP (Bisgrove et al., 2008) and TALIN-GFP ((Mathur et al., 1999) were used. In addition, the mutant ixr1-1 (Scheible et al., 2001) and prc1-1 (Desnos et al., 1996) were used.

Plant Expression Vectors.

3×Ypet-POM2/CSI1 was created using the basic experimental procedures described in (Zhou et al., 2011), by employing the TAC clone JAtY77F05 to generate an in frame C-terminal translational fusion between the CSI1/POM2 gene and the 3×Ypet tag (ABRC stock CD 1727). All of the Arabidopsis genomic sequences in the JAtY clone 77F05 10 Kb upstream and 5 Kb downstream of CSI1/POM2 were replaced by recombineering using the ampicillin- and tetracyclin-resistance genes, respectively. Primers used in this procedure and verification of insertion are shown in Table 1. The resulted plasmid was transformed into plants using GV3101 Agrobacterium tumefaciens standard protocols. T2 transformants were selected by BASTA (glufosinate ammonium 200 g/L) and the gene expression was validated by confocal microscopy.

TABLE 1
Primers sequence used in generation of 3xYpet-POM2/CSI1

| Usage | Name | Sequence |
|---|---|---|
| 1 | POM2 CF | 5′-GCAAAAGTGGTCCCAGAAACCTTGAGATAGAATTCCAGTGGT- |
| | | CTAACAAG-GGAGGTGGAGGTGGAGCT-3′ |
| | POM2 CR | 5′-TCGATTTAAGAAACCTCTCAAACAA- |
| | | AAACAAAAAATGAAGTGGTGGTTTAGGCCCCAGCGGCCGCAGCAG |
| | | CACC-3′ |
| 2 | POM2 CTF | 5′-TACAGTTTGAAAATTTGCCGG-3′ |
| | POM2 CTR | 5′-TGGGACAACAAAATGTTACAAATG-3′. |
| 3 | POM2 delRB | 5′-TCGCTGCTGGACATGCTCAGTTTGGTAAAGCCGGGGG- |
| | | GACCATTCTGAC-ttaccaatgcttaatcagtg-3′ |
| | replaRB- | 5′-TATATTGCTAATAAATTTTTGGCGCGCCGGCCAA- |
| | ampuniversal | TTAGGCCCGGGCGG-ttcaaatatgtatccgctcatg-3′, |
| | POM2 delLB | 5′- |
| | | TCTGATTAAATTTAATTAATATTATATTTTTAGAATCTTCTGAATA- |
| | | TTTA-ccctcttgggttatcaagagg-3′, |
| | replaLB- | 5′- |
| | tetuniversal | TTAGTTGACTGTCAGCTGTCCTTGCTCCAGGATGCTGTTTTTGACA |
| | | ACGG-taattcctaattatgttgacac-3' |

1: 3xYpet cassette upstream of the stop codon of CSI1/POM2
2: Test and sequence the insertion of the 3xYpet cassette
3: Used to generate this trimmed TAC clone

Chemical Treatments and Microscopy.

For microscopy, seedlings were grown as described above for 3 days and then transferred to 24-well plates containing 0.8 mL of 0.5×AGM with phytagar supplemented with either 0.5% (v/v) dimethyl sulfoxide (DMSO, Sigma), 15 μM CESTRIN (12 mM stock), or 30 μM oryzalin (40 mM stock) (Fisher (50-748-30). To ensure consistency, all treatments were carried out in phytagar supplemented media. Pulse treatments of Arabidopsis seedlings with the desired chemicals were performed for 2 hours in the dark.

Spinning disk confocal microscopy to observe the dynamics of CESA3-GFP×TUA5-RFP, 3×Ypet-POM2, and KORRIGAN-GFP (Vain et al., 2014) was performed using two similar customized microscopes (3I, Denver, Colo.) equipped with spinning disk heads (Yokogawa), EMMCCD cameras (Andor UK, Photometrics Tucson Ariz.), and oil immersion objectives (100×NA 1.4). Time-lapse images were taken every 5 seconds with exposure times between 800 ms and 600 ms.

Confocal images for CFP-SYP61-×CESA3-GFP were obtained using a Leica SP5 microscope using a 63× water objective employing dual channel sequential line scanning. Fluorescent markers were excited at 442 nm (CFP) and 488 nm (GFP). Additional confocal images were obtained using a Zeiss 710 equipped with a 63× oil and a 40× water objective. A 561 nm diode laser was used for propidium iodide (PI, Sigma) and RFP, a 488 nm for GFP, a 514 nm for YFP, and a 405 nm for CFP. Image analysis was performed using a combination of software tools: ImageJ software (version 1.36b; http://rsbweb.nih.gov/ij/), Image Pro Plus (Media Cybernetics, Rockville, Md.), and Imaris (Bitplane, Saint Paul, Minn.).

Hypocotyl Growth Measurements.

For growth analysis, seedlings were germinated on AGM containing the chemicals CESTRIN or isoxaben solubilized in DMSO and grown as described above. When the experiment was complete, the plates were scanned using a flatbed scanner (Epson Perfection V300) and hypocotyl lengths were measured using the segmented line tool in the image analysis software, ImageJ (Rasband et al., 1997-2014). Statistical analyses were carried out using the statistical package, R (R Core Team, 2012).

Yeast and E. coli Growth.

Single colonies of Saccharomyces cerevisiae-Y2H Gold and Schizosaccharomyces pombe were grown in liquid yeast extract-peptone-dextrose (YPD) media (10 g yeast extract, 20 g peptone, and 20 g glucose per 1 L of media, pH 7), at 30° C. for 24 hours. Yeast cultures were transferred to 3 mL of YPD to grow until an OD of 0.7 was reached. Ten μL of liquid YPD containing yeast at an OD 0.7 were placed onto solid YPD (10 g yeast extract, 20 g peptone 20 g glucose and 11 g bacto-agar per 1 L of media, pH 7) in a 6 well plate containing the chemicals. Four serial dilutions (1:10) were spotted per well. Plates were incubated at 30° C. for 3 days for Saccharomyces cerevisiae-Y2H Gold and 5 days for Schizosaccharomyces pombe and imaged with a flatbed scanner.

Two mL of Luria-Bertani (LB) media (10 g tryptone, 5 g yeast extract, and 10 g NaCl, pH 7) were inoculated with a single colony of TOP 10 cells and incubated overnight at 37° C. Thirty μL of this culture was then used to inoculate 3 mL of LB media containing the chemical. The OD was measured every 30 minutes over 8 hours using a spectrometer.

MT Polymerization Assay.

The MT polymerization data was obtained using a polymerization assay kit (Cytoskeleton, Inc. Denver Colo.), employing OD measurements of polymerized tubulin as previously described (Shelanski et al., 1973; Lee and Timasheff, 1977).

Cellulose Content.

Etiolated WT Col-0 Arabidopsis thaliana seedlings were grown on AGM containing CESTRIN as described above. Hypocotyls were harvested after 8 days of growth. Cell wall materials were isolated into alcohol insoluble residues (AIR) (Gunl et al., 2010) and cellulose content was estimated using a modified Updegraff method (Updegraff, 1969). Briefly, insoluble trifluoroacetic acid hydrolyzed AIR pellets were quantified using the Anthrone assay as described earlier Viles & Silverman (1949) and Dische (1962).

Phytophthora Growth

Phytophtora capsici inoculates were grown in corn agar media supplemented either with DMSO or different concentrations of CESTRIN. After 3 weeks cultures were imaged using flatbed scanner.

### Example 1

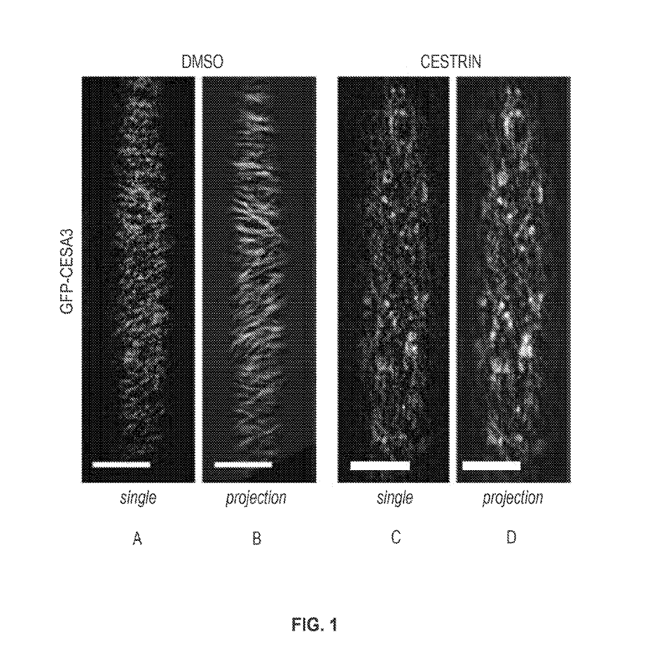

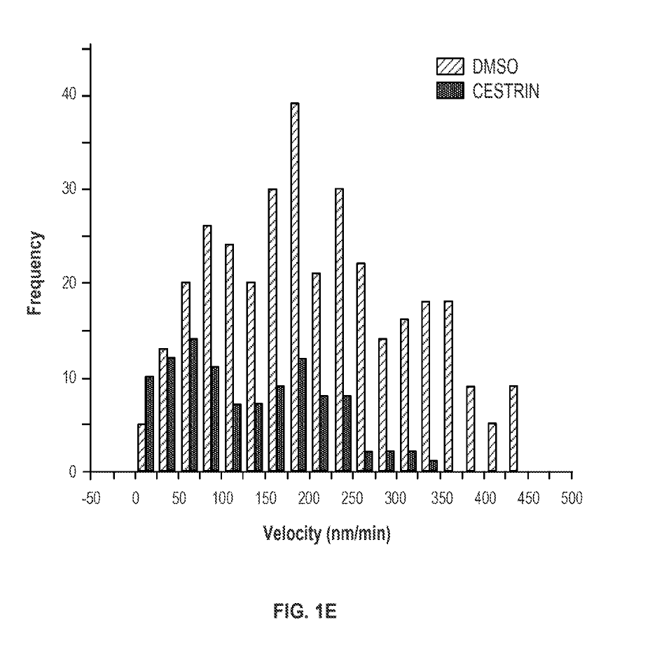

CESTRIN affects trafficking of cellulose synthase. Towards a better understanding of the CSCs trafficking, a library of 360 small molecules of pollen germination and endosomal trafficking inhibitors was screened (Drakakaki et al., 2011) for chemicals that specifically alter the localization of CESA in hypocotyls of three day-old etiolated Arabidopsis seedlings. A compound, 1-[2,6-dinitro-4-(trifluoromethyl)phenyl]-2-[6-methyl-4-(trifluoromethyl)pyridin-2-yl]hydrazine, Formula I, was identified that induces distinct and pronounced changes in the localization pattern of CSCs, as shown in FIG. 1. CESTRIN reduces GFP-CESA3 velocity (particle movement rate) and induces its accumulation in endomembrane compartments.

CSCs are enriched in SYP61 associated compartments upon CESTRIN treatment. The apparent redistribution of CSCs in the cell cortex prompted us to further investigate the identity of CESTRIN bodies. Previous studies have shown that CSCs are partially colocalized with SYP61/VHA-a1 in early endosome/TGN compartments (Crowell et al., 2009; Gutierrez et al., 2009). The presence of CESAs in SYP61 vesicles has been established by proteomic analysis (Drakakaki et al., 2012). Moreover, out of several endosomal/TGN markers previously investigated, SYP61 was shown to partially overlap with cortically tethered SmaCCs (Gutierrez et al., 2009). Hence, the behavior of GFP-CESA3 in relation to SYP61 under CESTRIN treatment was examined. Partial colocalization of the GFP-CESA3 and CFP-SYP61 compartments was observed under DMSO treatment, which was significantly enhanced (˜15%) upon CESTRIN application (p=0.0013, t-test). As previously shown, both SYP61 and SYP41/42 partially overlap with CESA6 under mock treatment, however only SYP61 remains partially colocalized after mannitol treatment (Gutierrez et al., 2009). Our data corroborate the presence of CSCs in SYP61 vesicles, which is increased by CESTRIN treatment.

Arabidopsis seedlings expressing GFP-CESA3 were grown in the dark for 3 days and imaged by spinning disk confocal microscopy. FIGS. 1A-B show seedlings expressing GFP-CESA3 were treated with DMSO (control). A single optical section and, for indication of motility, an average of 60 frames is shown. In FIGS. 1C-D, upon a 2-hour CESTRIN treatment, GFP-CESA3 particles no longer follow linear trajectories and are accumulated in punctae exhibiting increased fluorescence intensity. A single optical section and average of 58 frames are shown. The scale bar is 10 μm. In FIG. 1E, a histogram shows the distribution of GFP3-CESA3 velocities at the PM focal plane under DMSO (white) and CESTRIN (black) treatments.

In control, DMSO-treated plants, GFP-CESA3 follows linear trajectories at the PM as shown in FIGS. 1A-B. Contrasting this, in small molecule treated plants the overall number of CSCs at the PM was substantially reduced. Further, the particles showed reduced motility as demonstrated in the translocation tracks of time-projected images as shown in FIGS. 1C-D. Analysis of time-lapse confocal imaging sequences revealed that the average velocity of the particles dropped from 218 nm/min in the control sample to 127 nm/min in the treated sample as shown in FIG. 1E. Concurrently, much of the GFP-CESA3 accumulated in compartments at the cell cortex, exhibiting increased fluorescence intensity and larger size compared to the control. It is likely that this subcellular population contains the CSCs in SmaCCs/MASCs compartments. Given the effect on CESA trafficking inhibition, the compound is referred to as “CESTRIN” and the increased fluorescence compartments as “CESTRIN induced bodies.”

### Example 2

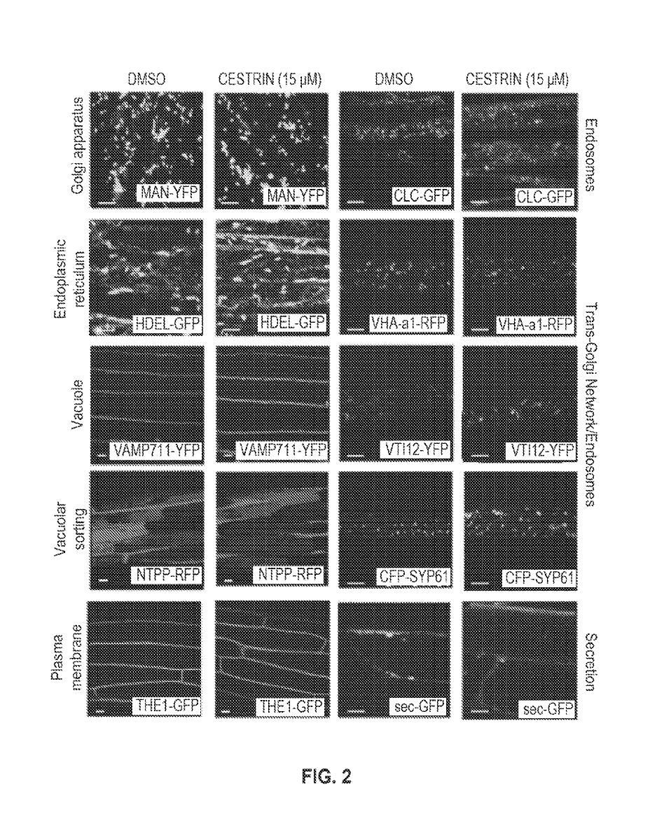

In order to assess the specificity of CESTRIN, a variety of organelle markers and their subcellular localizations were examined in Arabidopsis etiolated hypocotyls. As shown in FIG. 2, the overall morphology of the ER, Golgi, TGN, early endosomes (E/E), and vacuole and the trafficking of soluble cargo to the vacuole were not noticeably affected, indicating that CESTRIN does not exert broad toxicological effects. The unaltered morphology of endosomes labeled by CLC2-GFP demonstrates that CESTRIN does not target endocytic trafficking and is selective (see FIG. 2). Further, neither the localization of the PM-receptor-like kinase Theseus-GFP nor the secretion marker sec-GFP were altered (see FIG. 2), suggesting that the drug does not broadly disrupt trafficking to the plasma membrane or secretion. Taken together these results suggest that CESTRIN perturbs the localization and the motility of compartments involved in CSCs trafficking.

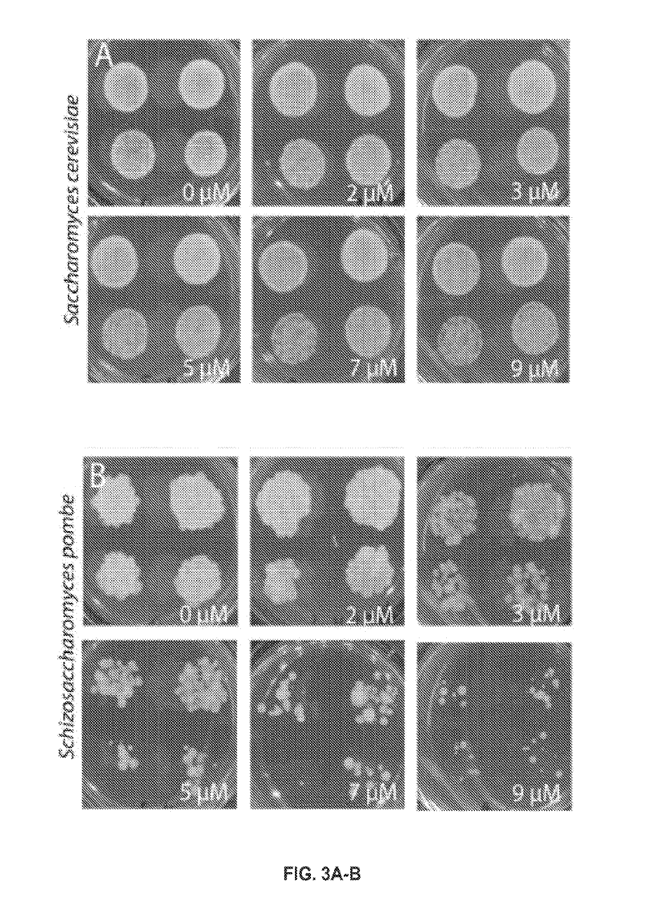

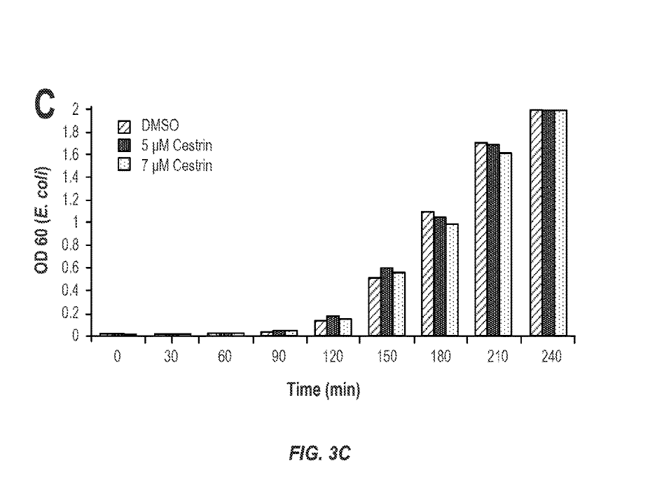

CESTRIN activity and its mode of action is conserved across plants and yeast. In order to evaluate the effect of CESTRIN on different organisms, the growth of bacteria (E. coli) and two species of yeast cells (Saccharomyces cerevisiae and Schizosaccharomyces pombe) was analyzed. Evaluating whether CESTRIN causes a broad growth inhibition, its impact on bacteria (E. coli) was analyzed. As shown in FIG. 3, no effect on their proliferation was observed; however, the growth of yeast Schizosaccharomyces pombe was inhibited under CESTRIN treatment.

### Example 3

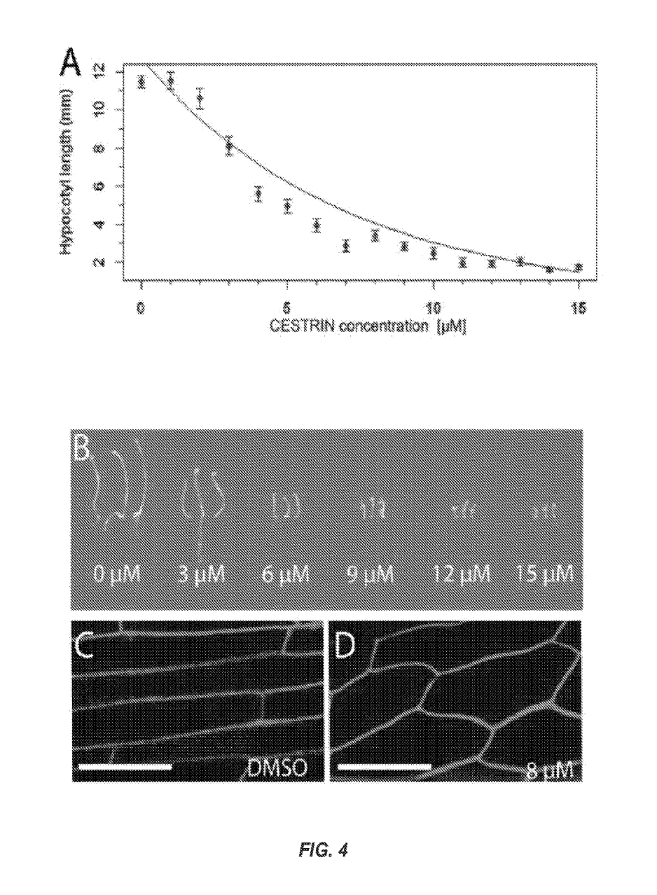

CESTRIN inhibits cell elongation and reduces cellulose content. CESTRIN inhibits anisotropic growth in Arabidopsis. FIGS. 4A-B show concentration dependent growth inhibition of 5-day-old Arabidopsis etiolated hypocotyls under CESTRIN treatment. The half maximal inhibitory concentration (IC50) is calculated to be 4.85 mM using an exponential trendline, R²=0.9416, n=48. FIGS. 4C-D show propidium iodide staining of hypocotyl cells in 5-day-old Arabidopsis seedlings treated with CESTRIN, which show decreased elongation and increased radial swelling. The scale bar is 50 μm.

CESTRIN reduces anisotropic cell growth in a concentration dependent manner with an estimated IC₅₀ of 4.9 μM as shown in FIGS. 4A-B. It induces cell shape morphological changes in hypocotyls with clearly visible cell swelling and reduced cell elongation, as shown in FIGS. 4C-D. Arabidopsis hypocotyls grown on 9 μM CESTRIN contained ˜30% less cellulose compared to control seedlings, as shown in Table 2.

TABLE 2
CESTRIN treatment significantly reduces cellulose
content in Arabidopsis etiolated seedlings
Cellulose content, μg/mg AIR

| Treatment | Hypocotyl |
|---|---|
| DMSO | 103.7 ± 6.4 |
| CESTRIN (9 μM) | 75.3 ± 2.7* |

### Example 4

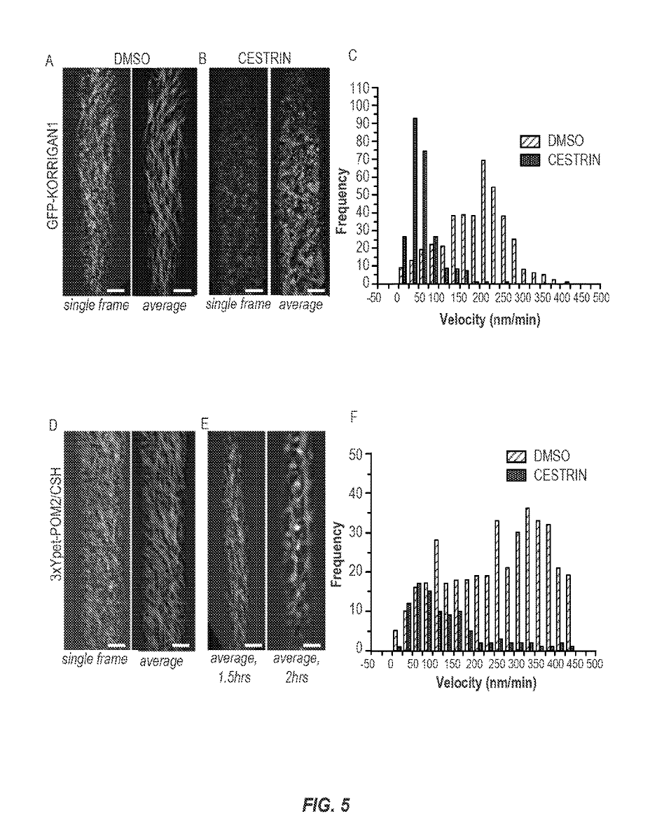

CESTRIN alters the localization velocity of proteins interacting with CSCs GFP-KOR1 and 3×Ypet-POM2/CSI1. CESTRIN treatment induces mislocalization of the CESA interacting proteins POM2/CSI1 and KOR1. Recent studies have shown that the glucanase KOR1 is an integral part of the primary cell wall CSCs at the plasma membrane. Similar to CSCs, its localization pattern follows microtubule reorientation during epidermal cell elongation (Lei et al., 2014b; Vain et al., 2014). Whether CESTRIN affects CESAs or KOR1 in a differential manner was analyzed by comparing the respective localization patterns. Under control conditions, GFP-KOR1-labelled plasma membrane particles migrate along linear trajectories with comparable velocities (average of 220 nm/min) as those observed for GFP-CESAs, as shown in FIGS. 5A & 5D. Overall, the trafficking pattern of CSCs and KOR1 showed similar behavior after CESTRIN treatment. However, subtle differences were also evident. CESTRIN treatment dramatically reduced the presence of moving GFP-KOR1 at the PM, and instead concentrated the protein in trafficking compartments at the cell cortex as judged from the higher fluorescence intensity, as shown in FIGS. 5B & 5E. The mean velocity of GFP-KOR1 labelled particles was drastically reduced to ˜60 nm/min, as shown in FIG. 5F, with particle motion resembling a random walk rather than a straight translocation.

Arabidopsis seedlings expressing GFP-KOR1 and 3×Ypet-POM2/CSI1 were grown in the dark for 3 days and imaged by spinning disk confocal microscopy. In FIG. 5A, seedlings expressing GFP-KOR1 were treated with DMSO (control). A single optical section and an average of 50 frames is shown. In FIG. 5B, upon a 2-hour CESTRIN treatment, GFP-KOR1-particles are accumulated in punctae exhibiting increased fluorescence intensity. A single optical section and an average of 55 frames are shown. The scale bar is 5 μm. In FIG. 5C, a histogram depicts the frequency of GFP-KOR1 velocities at the PM focal plane under DMSO (white) and CESTRIN (black) treatments.

CESAs involved in primary cell wall biosynthesis are interacting with CSI/POM2, (Gu et al., 2010; Bringmann et al., 2012a); this prompted investigation into the trafficking dynamics of 3×Ypet-POM2 under chemical treatment in relation to CESAs. In control plants the localization pattern of 3×Ypet-POM2 showed distinct punctae that exhibit a directional motility, as shown in FIG. 5B, in accordance with previous observations (Gu et al., 2010; Bringmann et al., 2012a). However, in chemically treated plants, 3×Ypet-POM2-labeled particles lost organization, leading to a more dispersed localization, concurrent with a reduction in directional movement after 1.5 hours of treatment, as shown in FIG. 5C. After 2 hours of treatment, diffused patterns of the fluorescent fusion were observed, suggesting cytoplasmic dissociation of POM2 from the plasma membrane CSCs. The average motility of POM2 labelled particles was reduced from ˜262 nm/min (n=420) to 164 nm/min n=106 after about 1.5 hours of treatment and the particles came virtually to a rest after 2 hours, as shown in FIG. 5D. Overall these data suggest that CESTRIN inhibits the dynamics and integrity of CESA complexes with POM2!CSI-1 and demonstrate their altered localization patterns. In FIG. 5D, seedlings expressing 3×Ypet-POM2/CSI1 were treated with DMSO (control). A single optical section and an average of 58 frames is shown. In FIG. 5E, upon a 1.5 hour CESTRIN treatment, 3×Ypet-POM2 particles show altered distribution pattern. An average of 60 frames is shown. Upon 2 hours of CESTRIN treatment, 3×Ypet-POM2 was localized to the cytoplasm. An average of 60 frames is shown. The scale bar is 5 μm. In FIG. 5F, a histogram depicts the frequency of 3×Ypet-POM2 velocities at the PM focal plane under DMSO (white, n=420) and CESTRIN (black, n=106) treatments.

### Example 5

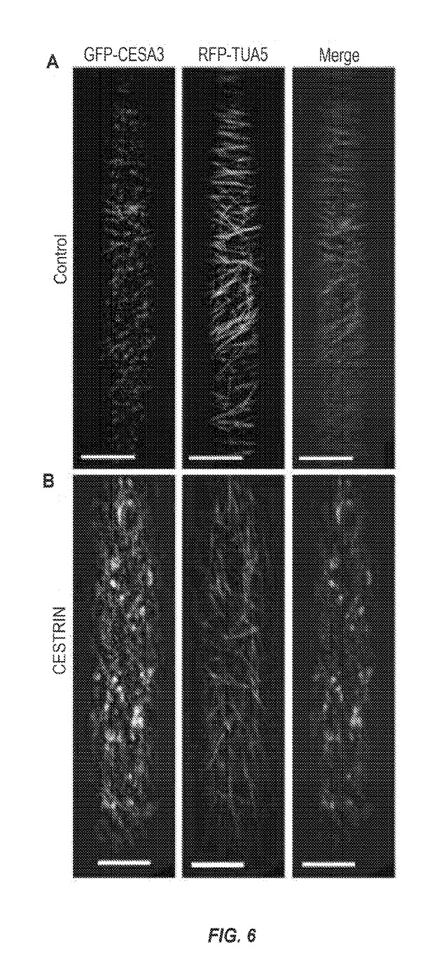

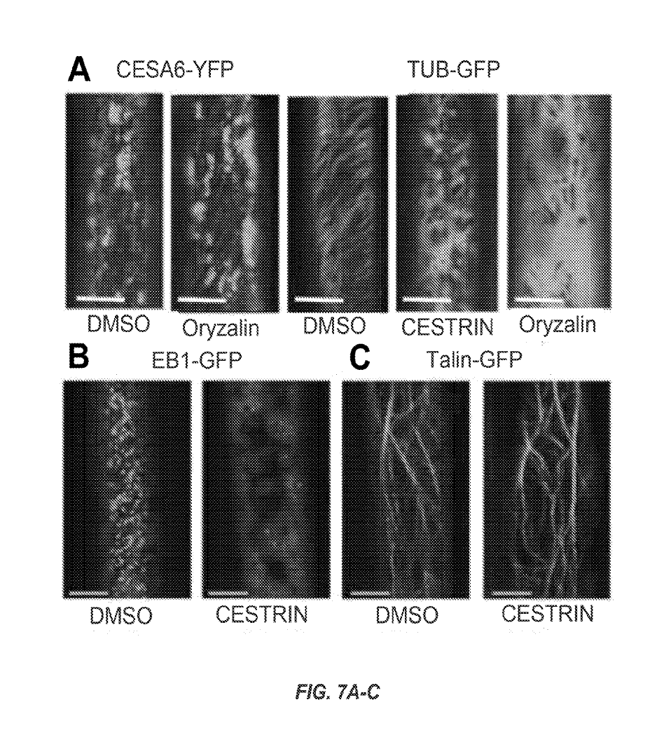

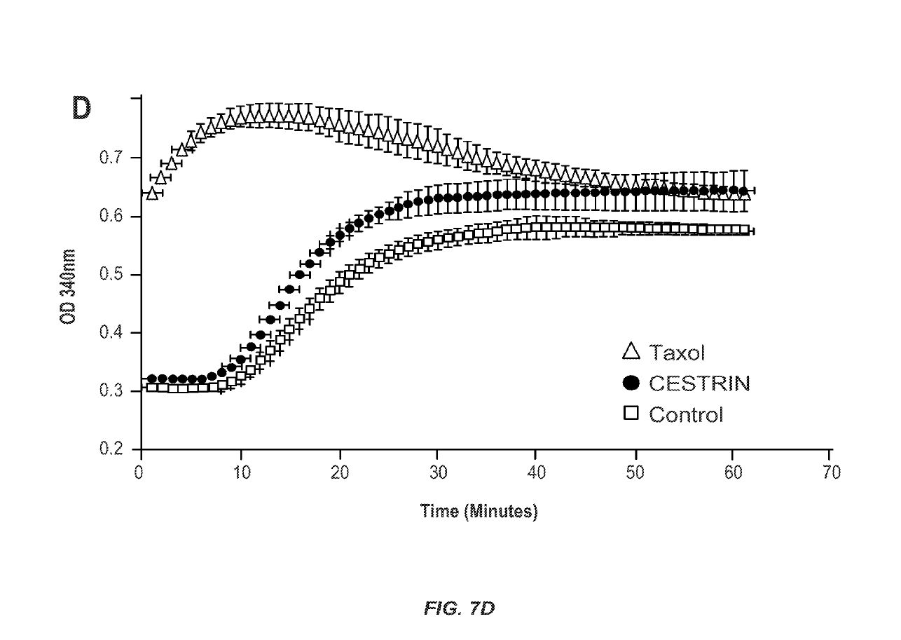

CESTRIN alters MT stability in a mechanism different from oryzalin. Given that CESAs interact closely with MTs, the effect of CESTRIN on MT stability in relation to CESA localization was studied using Arabidopsis seedlings expressing GFP-CESA3/mCherry-TUA5 (Gutierrez et al., 2009). Concurrent with pronounced mislocalizations of GFP-CESA3, CESTRIN treatment induced several marked changes in microtubule organization, including the reduction of clear transverse-oriented cortical arrays in comparison with DMSO-treated controls, as shown in FIGS. 6A, 6B, and 7A. Three-day-old etiolated Arabidopsis seedlings expressing both RFP-TUA5 and GFP-CESA3 were imaged. FIG. 6A shows DMSO treated seedlings. Trace from 6 frames shows colocalization of GFP-CESA3 particles and MTs. FIG. 6B shows a two-hour CESTRIN treatment causes loss of MT organization and redistribution of GFP-CESA3 particles, trace composed of 58. The scale bar is 10 μm.

The majority of the treated cells featured disordered MT arrays or more diffuse fluorescent patterns (FIGS. 6A, 6B, and 7A). CESAs are typically delivered to sites that coincide with cortical MTs (Gutierrez et al., 2009). A possible explanation for the observed CESA behavior could be a failure to deliver CESAs to MT sites, and/or an inability to properly guide the CSCs along the MTs. This hypothesis in turn prompted comparison of CESTRIN with the microtubule depolymerization drug oryzalin, which binds tubulin dimers and interferes with the dimer addition to microtubule ends (Morejohn et al., 1987; Chan et al., 2003). The effect of CESTRIN on cortical MT array organization shares partial similarities with oryzalin. As mentioned above, CESTRIN treatment caused alterations in organization and depolymerization of MTs, leading to less defined arrays and a more diffused pattern of presumably free mCherry-TUA5 (FIGS. 6B and 7A). This effect is similar for oryzalin but is, however, less pronounced. A dramatically different behavior is observed for the cellular dynamics of CSCs during CESTRIN and oryzalin treatments. Even under extended oryzalin treatment, the CSCs do not dissociate from the PM, whereas this is seen during CESTRIN treatments (FIG. 7) (Paredez et al., 2006; Gutierrez et al., 2009).

FIG. 7A shows Oryzalin (30 μM) treatment of etiolated Arabidopsis hypocotyls showed no morphological changes in Cellulose Synthase 6 (CESA6-YFP) and depolymerization of tubulin (TUB-GFP), demonstrating differences between the MT depolymerizing drug and CESTRIN. FIG. 7B shows CESTRIN (15 JIM) induced cytoplasmic localization of the end binding protein 1 (EB1-GFP). FIG. 7C shows CESTRIN (15 μM) does not affect the morphology or organization of actin (Talin-GFP). FIG. 7D shows in-vitro polymerization of tubulin is not affected by 15 μM CESTRIN.

To further investigate the impacts of CESTRIN on the MT arrays, the localization pattern of the MT end plus binding protein (EB1) (Dixit et al., 2006) was examined. As previously described, GFP-EB1 localizes in distinct foci that dominantly label MT plus ends (FIG. 7B). In contrast, CESTRIN treated cells showed diffuse EB1 fluorescence in the cytoplasm (FIG. 7B). It is likely that the loss of MT stability due to CESTRIN leads to the loss of MT localized EB1. To further investigate if CESTRIN is a direct MT polymerization inhibitor, an in vitro spectrometric absorption assay was employed. The assay did not yield any evidence for CESTRIN affecting the polymerization process (FIG. 7D), making this an unlikely targeted action of the small molecule. In stark contrast to the distinct changes in the microtubules, no changes were observed for actin organization and dynamics under chemical treatment (FIG. 7C), which further demonstrates that the small molecule does not broadly affect the cytoskeleton.

### Example 6

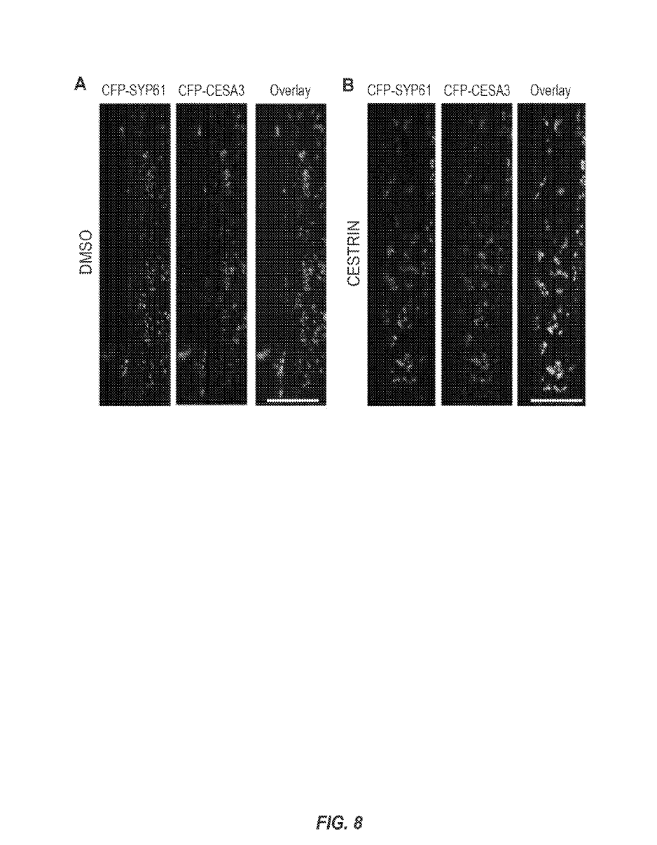

Isoxaben-resistant plants are not cross-resistant to CESTRIN. To determine if CESTRIN has the same mechanism of action, as the well characterized cellulose inhibitor isoxaben, the hypocotyl growth in etiolated seedlings of the isoxaben-resistant CELLULOSE SYNTHASE 3 mutant ixr1-1 was compared with that of WT Col-0 and the CELLULOSE SYNTHASE 6 prc1-1 allele. FIG. 8A shows hypocotyl growth of the WT Col-0, the isoxaben-resistant CESA3 mutant ixr1-1, and the CESA6 mutant prc1-1 in 8 μM CESTRIN compared to in DMSO supplemented media (A) (n=16 per treatment). FIG. 8B shows wild type Col-0, ixr1-1, and prc1-1 5-day-old seedlings grown on media containing either 4 nM isoxaben or DMSO (n=16 per treatment). The hypocotyl growth of ixr1-1 was reduced under CESTRIN treatment by 62% (p=8.6×10-5), while it was not reduced under isoxaben treatment (p=0.2) when compared to the WT Col-0. When comparing Col-0 and ixr1-1, the two genotypes showed significantly different responses to the two chemicals (Two-way ANOVA p=4.7×10-12). Significantly different response of prc1-1 compared to Col-0 was observed for both chemical treatments (Two-way ANOVA p=0.00⁸).

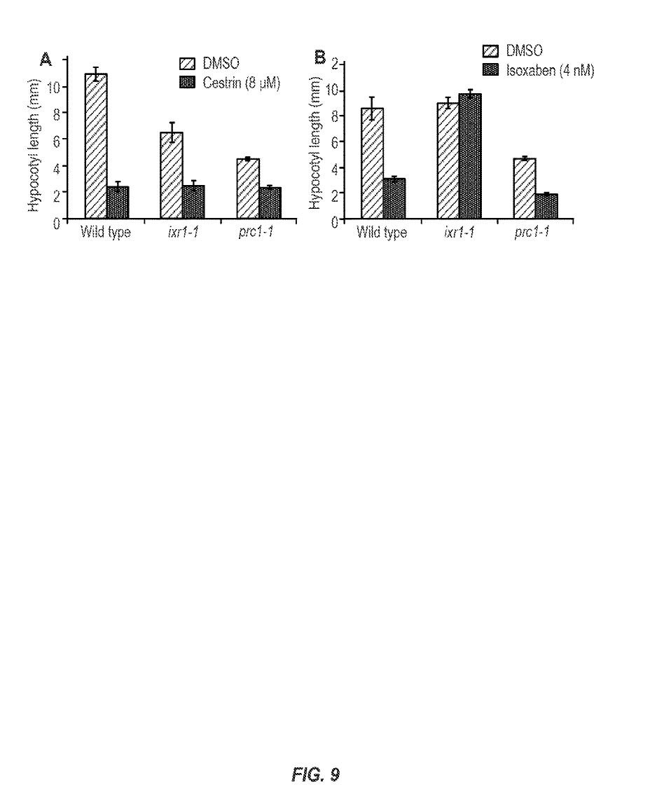

While the hypocotyl growth of ixr1-1 is not reduced under isoxaben treatment (P=0.2), a 62% reduction was observed for CESTRIN treatment (P=8.6×10⁻⁵); however, the reduction was more pronounced for the WT Col-0, exhibiting an 80% reduction (P=0.001). When comparing Col-0 and ixr1-1, the two genotypes showed significantly different responses to the two chemicals (Two-way ANOVA P=4.7×10⁻¹²). In addition, a significantly different response of prc1-1 compared to Col-0 was observed for both chemical treatments (Two-way ANOVA P=0.008). The prc1-1 mutant showed sensitivity to both isoxaben and CESTRIN, however to a lower degree, compared to the growth reduction in the WT Col-0. Taken together, these data establish that isoxaben and CESTRIN are acting on unique targets. FIGS. 9A-B are charts comparing hypocotyl growth of ixr1-1 and prc1-1 in CESTRIN compared to DMSO and on media containing 4 nM isoxaben or DMSO.

### Example 7

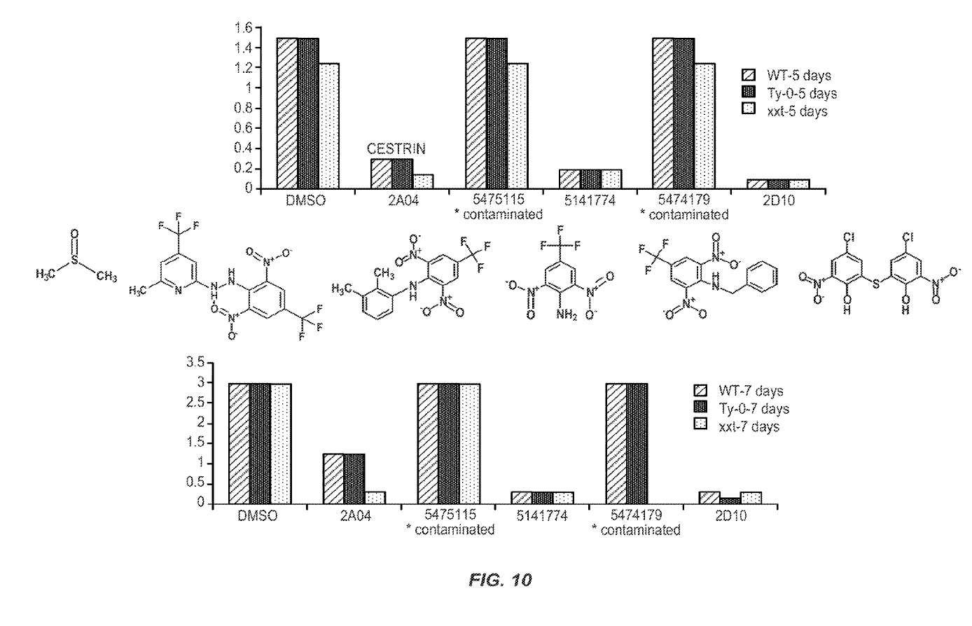

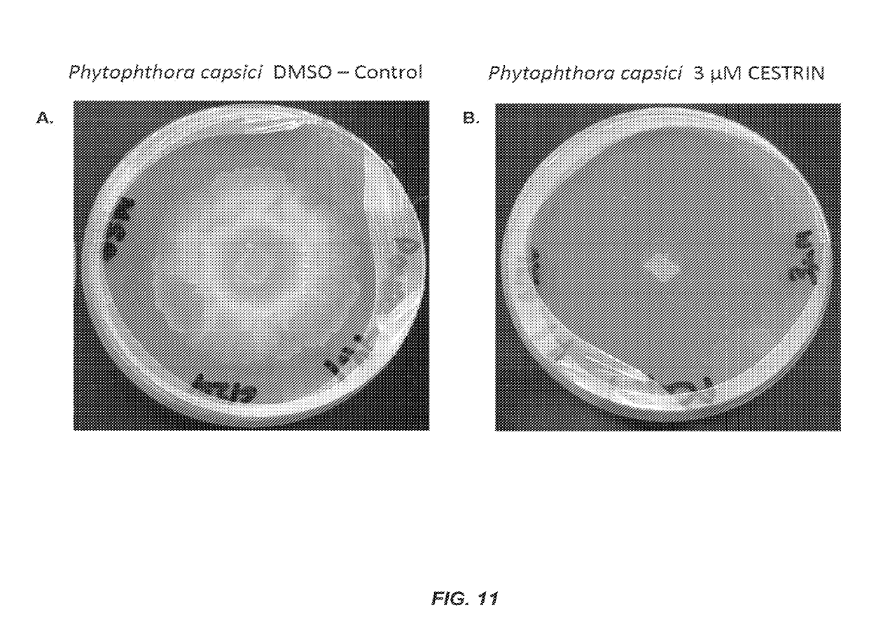

Root growth experiments. For growth analysis, seedlings were germinated on AGM containing the chemicals CESTRIN or analogues solubilized in DMSO and grown vertically in the light for 7 days. When the experiment was complete, the plates were scanned using a flatbed scanner (Epson Perfection V300) and root lengths were measured using the segmented line tool in the image analysis software, ImageJ (Rasband et al., 1997-2014). Results are shown in FIG. 10.

### Example 8

CESTRIN inhibits phytophtora capsici growth Phytophthora cultures were grown for 3 weeks in media supplemented with DMSO control or CESTRIN. FIG. 11A shows Phytophtora capsici cultures grown in media supplemented with DMSO (control). FIG. 11B shows Phytophthora capsici growth is completely inhibited under CESTRIN treatment.
Bringmann, et al. (2012). POM-POM2/cellulose synthase interacting1 is essential for the functional association of cellulose synthase and microtubules in Arabidopsis. Plant Cell 24, 163-177.
Crowell, et al. (2009). Pausing of Golgi bodies on microtubules regulates secretion of cellulose synthase complexes in Arabidopsis. Plant Cell 21, 1141-1154.
Drakakaki, et al. (2012). Isolation and proteomic analysis of the SYP61 compartment reveal its role in exocytic trafficking in Arabidopsis. Cell research 22, 413-424.
Drakakaki, et al. (2011). Clusters of bioactive compounds target dynamic endomembrane networks in vivo. Proc Natl Acad Sci USA 108, 17850-17855.
Groen, et al. (2014). Identification of trans-golgi network proteins in Arabidopsis thaliana root tissue. J Proteome Res 13, 763-776.
Gu, et al. (2010). Identification of a cellulose synthase-associated protein required for cellulose biosynthesis. Proc Natl Acad Sci USA 107, 12866-12871.
Gutierrez, et al. (2009). Arabidopsis cortical microtubules position cellulose synthase delivery to the plasma membrane and interact with cellulose synthase trafficking compartments. Nature Cell Biol. 11, 797-806.
Paredez, et al. (2006). Visualization of cellulose synthase demonstrates functional association with microtubules. Science 312, 1491-1495.
Vain, et al. (2014). The cellulase KORRIGAN is part of the Cellulose Synthase Complex. Plant Physiol.

All patents, patent applications, and publications cited herein are hereby incorporated by reference in their entireties.

1. An herbicidal or fungicidal composition comprising an effective amount of a compound of Formula I, II, III, IV, V, VI, VII, VIII, IX, X, XI, XII or XIII.

2. The composition of claim 1, wherein the composition is an herbicidal composition.

3. The composition of claim 2, wherein the herbicidal composition further comprises at least one of a solvent, a surfactant, and a dispersing medium.

4. The composition of claim 2, wherein the composition is in the form of an emulsion or emulsifiable liquid concentrate.

5. The composition of claim 2, wherein the composition includes a solid carrier.

6. The composition of claim 5, wherein the composition is in the form of a wettable powder or a paste.

7. The composition of claim 1, wherein the composition is a fungicidal composition.

8. The composition of claim 7, wherein the composition further comprises at least one of a solvent, a surfactant, and a dispersing media.

9. The composition of claim 7, wherein the composition is in the form of an emulsion or emulsifiable liquid concentrate.

10. The composition of claim 7, wherein the composition includes a solid carrier.

11. The composition of claim 10, wherein the composition is in the form of a wettable powder or a paste.

12. A method of controlling unwanted vegetation, the method comprising applying an effective amount of the composition of claim 1.

13. A method of controlling unwanted fungus, the method comprising applying an effective amount of the composition of claim 1.

14. A method of inhibiting cellulose biosynthesis, the method comprising applying an effective amount of the composition of claim 1.

15. A method of inhibiting cellulose biosynthesis, the method comprising applying an effective amount of the composition of claim 1.
